(19) United States
(12) Patent Application Publication
(10) Pub. No.: US 2022/0375088 A1
(43) Pub. Date: Nov. 24, 2022

(54) MAGNETIC RESONANCE (MR) IMAGE ARTIFACT DETERMINATION USING TEXTURE ANALYSIS FOR IMAGE QUALITY (IQ) STANDARDIZATION AND SYSTEM HEALTH PREDICTION

(71) Applicant: KONINKLIJKE PHILIPS N.V., EINDHOVEN (NL)

(72) Inventors: Tejas Jatin Shah, Bangalore (IN); Anasuya Mohan Rao, Bangalore (IN); Paul Royston Harvey, Best (NL)

(21) Appl. No.: 17/765,468

(22) PCT Filed: Oct. 2, 2020
(86) PCT No.: PCT/EP2020/077700

§ 371 Date: Mar. 31, 2022

Related U.S. Application Data
(60) Provisional application No. 62/910,504, filed on Oct. 4, 2019.

(51) Int. Cl. G06T 7/00; A61B 5/00; A61B 5/055; G06T 7/41; G06V 10/54; G06V 10/70; G16H 30/40; G16H 40/63

(52) U.S. Cl. CPC G06T 7/0014; A61B 5/7221; A61B 5/055; A61B 5/742; G06T 7/41; G06V 10/54; G06V 10/70; G16H 30/40; G16H 40/63; A61B 2576/00; G06T 2207/20081; G06T 2207/30168; G06T 2207/30004; G06T 2207/10088

(57) ABSTRACT

An apparatus (100) comprises at least one electronic processor (101, 113) programmed to: control an associated medical imaging device (120) to acquire an image (130); compute values of textural features (132) for the acquired image; generate a signature (140) from the computed values of the textural features; and at least one of: display the signature on a display device (105); and apply an artificial intelligence (AI) component (150) to the generated signature to output image artifact metrics (152) for a set of image artifacts and display an image quality assessment based on the image artifact metrics on the display device.

### FIELD

The following relates generally to the imaging device servicing and maintenance arts, especially as directed to medical imaging device servicing or the servicing of other complex systems, maintenance history analysis arts, artificial intelligence (AI) arts, and related arts.

### BACKGROUND

An important commodity in diagnostic imaging is the image quality (IQ). In practice, vendors use internationally accepted standardized tests such as the American College of Radiology (ACR) or the National Electrical Manufacturer's Association (NEMA) and/or vendor-specific customized procedures, for IQ assessment. However, an underlying assumption of such methods is the presence of a handful of image artifacts. Additional image acquisitions are required to address these artifacts, which can use very specific image acquisition protocols and specific phantoms whose set up necessitates additional execution time. Moreover, such methods require extensive user skills and expertise in selecting the appropriate acquisition protocols, correctly setting up the apparatus, quantification of degraded IQ, interpretation of the images, choice of tools/methodology used for detection and interpretation of computed quantitative results. Due to such reliance on user skill and expertise, such methods are very subjective.

The images acquired from a medical imaging device has a wealth of information that could be harnessed to get insights into system performance itself. If such information is available, it would allow one to monitor the health status of the system and/or component, predict failure, provide predictive maintenance and also allows control over wider range of sources that could affect the IQ. Current methods are not capable of capturing this information without installation of additional sensors or monitoring devices.

Utilizing current methods to find the root cause of poor IQ requires considerable skill and expertise. Even with skill and expertise, the process is iterative and possibly might not lead to a particular root cause making the entire process very laborious, ineffective, and time & resource consuming for service vendors as well as its customers. Moreover, these methods are designed to detect only a few select artifacts.

Although metrics computed based on current methods could be archived, limitations of such methods are that they are laborious, require additional execution time, only captures large fluctuations in image quality and cannot point towards a possible root cause for poor IQ. Due to these limitations, the ability to monitor system or component health over time is very restricted.

The following discloses certain improvements to overcome these problems and others.

### SUMMARY

In one aspect, an apparatus comprises at least one electronic processor programmed to: control an associated medical imaging device to acquire an image; compute values of textural features for the acquired image; generate a signature from the computed values of the textural features; and at least one of: display the signature on a display device; and apply an AI component to the generated signature to output image artifact metrics for a set of image artifacts and display an image quality assessment based on the image artifact metrics on the display device.

In another aspect, a service device includes a display device; at least one user input device; and at least one electronic processor programmed to: compute values of textural features from an image from an image acquisition device undergoing service; generate image artifact metrics for a set of image artifacts from the computed values of the features; and control the display device to display an image quality assessment based on the image artifact metrics.

In another aspect, an image quality deficiency identification method includes: acquiring one or more clinical images over a periodic temporal period using an image acquisition device; computing at least one textural feature for the acquired at least one image; analyzing patterns in the computed at least one textural feature via a signature generated from the at least one textural feature over time to predict a potential issue with the image acquisition device.

One advantage resides in providing a turnkey solution for IQ assessment, which can be used by an imaging technologist, field service engineer, or other user without specialized training in order to provide a quantitative assessment of various types of artifacts impacting IQ.

Another advantage resides in reducing costs for monitoring imaging system health and planning of maintenance or servicing visits, as well as reducing warranty costs.

Another advantage resides in providing an objective IQ standard to determine IQ of images.

Another advantage resides in providing IQ assessment with reduced interruptions in customer (i.e. end user) productivity.

Another advantage resides in providing automated identification of a ranked list of types of image artifacts present in images produced by an imaging system.

Another advantage resides in providing automated identification of an underlying root cause of image artifacts.

Another advantage resides in detecting fine IQ fluctuations from images acquired during a route quality assessment period.

Another advantage resides in using multiple texture features to allow detection and differentiation between different artifacts.

Another advantage resides in the use of multiple texture signatures that differentiates the use of IQ assessment using texture analysis.

Another advantage resides in utilizing pattern recognition and machine learning algorithm to identify various artifacts and their root causes.

Another advantage resides in using textural feature signatures for IQ assessment more robust and reproducible.

Another advantage resides in providing an automated or semi-automated way to detect and identify different artifacts in a user-friendly manner.

Another advantage resides in allowing tighter control over IQ, thus allowing tighter control over an IQ standard across an MR fleet.

Another advantage resides in archiving texture features indicative of artifacts impacting IQ in order to trend corresponding values to allow system IQ monitoring and/or predicting system failures.

Another advantage resides in enabling improvements in medical imaging system reliability over a period of time through detection and identification of artifacts and their source.

Another advantage resides in increasing medical imaging system uptime through prediction of medical imaging system component failure.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative apparatus for image quality assessment in accordance with the present disclosure.

FIG. 2 shows exemplary flow chart operations of the system of FIG. 1.

FIGS. 3A and 3B show examples of generated signature for image artifacts.

FIG. 4 shows exemplary flow chart operations of the system of FIG. 1.

### DETAILED DESCRIPTION

The following pertains to an improved image quality (IQ) assessment. IQ relates to the presence/strength of various artifacts, which may be localized or uniform across the image. Existing IQ assessments typically employ imaging of a phantom followed by some subjective visual IQ assessment by an imaging expert, and/or applying some IQ assessment algorithm.

In some embodiments disclosed herein, the IQ assessment is performed by computing textural features of the image. “Generally speaking, textures are complex visual patterns composed of entities, or subpatterns, that have characteristic brightness, color, slope, size, etc. Thus texture can be regarded as a similarity grouping in an image. The local subpattern properties give rise to the perceived lightness, uniformity, density, roughness, regularity, linearity, frequency, phase, directionality, coarseness, randomness, fineness, smoothness, granulation, etc., of the texture as a whole.” Materka et al., “Texture Analysis Methods—A Review”, Technical University of Lodz, Institute of Electronics, COST B11 report, Brussels 1998. In some illustrative embodiments, the texture features defined in Haralick et al., “Textural Features for Image Classification”, IEEE Trans. on Systems, Man, and Cybernetics vol. SMC 3 no. 6 (1973) are used. A signature constructed from a number of these Haralick (or other) textural features is effective for discriminating whether an image has artifacts arising from the medical imaging system or the medical imaging system environment. Some examples of such artifacts can include spike noise, artifacts arising due to malfunctioning of components in a transmission-receiving chain including radiofrequency (RF) artifacts, such as RF interference noise, or RF coil-related artifacts, among others. In this way, a turnkey solution is provided by which an imaging technologist, field service engineer, or other user without specialized training can quickly and quantitatively assess various types of artifacts impacting IQ.

The IQ assessment tool can be used to acquire an image of a standard phantom (or, in some other approaches, a clinical image of a patient), compute the standard textural features for the image, generate a signature from the textural feature values, and provide IQ analysis based on the signature. One possible signature is a spider plot comparing the image with a baseline normal image. The analysis is suitably performed by inputting the signature into a trained artificial intelligence (AI) component (such as machine learning component or a deep learning component) that outputs metrics of various artifacts and may identify a ranked list of artifact(s), possibly along with its root cause(s), e.g., retrieved from a look-up table associating artifacts (or combinations of artifacts) with root causes.

In the textural features of Haralick et al., the textural feature extraction involves two steps. First, gray level co-occurrence matrices (GLCM) are computed for the image. Each GLCM is computed for directions quantized to 45° intervals, and is an N×N matrix where N is the number of gray levels. The GLCM is parameterized by distance d which is the distance along the designated direction separating the two pixels being compared. For example, if the direction is 45° (that is, to the upper right) and d=5, then the matrix element (120, 125) would store a (typically normalized) count of the co-occurrences in the image of a pixel with gray level 120 and a pixel with gray value 125 being 5 positions up and to the right of the pixel valued 120. A small value for d is expected to be sufficient (e.g., 2), and has an advantage in improving computational speed. The textural features are then computed from the GLCMs (see, e.g. Haralick et al.), and are scalar values. Hence, if there are, e.g., 15 textural features then the image is characterized by 15 real-valued textural feature values. While Haralick textural features are used herein as illustrative examples, other types of textural features may additionally or alternatively be used.

The AI component is suitably trained on training images of the standard phantom, which are manually labeled as to artifact metrics by imaging experts so as to provide ground truth labels. This may be a one-time training phase for a given imaging modality (or, possibly, a given imaging device model), with the trained AI component then being shipped to customers. It is also contemplated to train the AI component on clinical images of actual patients, although variation of image content between patients may make this approach less robust. Yet another approach is to train on a training set including a mixture of phantom images and clinical images.

In some embodiments disclosed herein, the IQ assessment tool is provided as a web service, and/or an application program (“app”) running on a tablet computer, cellphone, or other mobile device carried by a field service engineer (FSE) or on the scanner controller, or so forth. The FSE carries the standard phantom (or one is stored at the customer site) and acquires images of it using the imaging device undergoing service, preferably using a standard IQ assessment imaging sequence. (Alternatively, recently acquired clinical images may be used). These images are input to the IQ assessment tool which identifies a list of artifact(s) along with their root cause(s) and possibly a recommended repair. After performing the repair, the imaging and IQ assessment is repeated to determine whether the problem has been solved.

The standard IQ assessment imaging sequence should be the same imaging sequence that was used to acquire the training images of the standard phantom used to train the AI component, or at least a similar imaging sequence to the one used in the training. The detailed design of the standard IQ assessment imaging sequence can vary, but it is preferably representative of a typical medical imaging task performed by the medical imaging system. For example, the standard IQ assessment imaging sequence preferably uses all gradient coils, preferably over their usual operating range of gradient coil electric currents, preferably uses the RF coil or set of RF coils and/or RF coil arrays used in imaging patients, preferably employs imaging field of view (FOV) and resolution typically used when imaging patients, and so forth. If the imaging device is used for a wide range of different imaging tasks (e.g. whole body imaging, brain imaging, limb imaging, magnetic resonance angiography, and/or so forth) then two or more different IQ assessment imaging sequences may be employed in order to represent the full operational envelope of the imaging system. In this case, a separate AI component is suitably used to analyze IQ of the image produced by each IQ assessment imaging sequence, with that AI component suitably trained on artifact-labeled training images acquired using that IQ assessment imaging sequence. In some embodiments, the standard IQ assessment imaging sequence may be a clinical imaging sequence, which may facilitate using clinical images recently acquired using that clinical sequence as the images input to the AI component to perform the IQ assessment.

In some embodiments disclosed herein, an image of the phantom is acquired occasionally (e.g., once-a-day or once-a-week) and the IQ assessment tool is run on the image. The textural features are archived. A trained AI component analyzes the trends in the textural features over time to predict a system or component failure, and this prediction is provided to the customer or to a maintenance staff to schedule proactive maintenance.

In this embodiment, the AI component is suitably trained on data collected at customer sites over time. For example, the customer may be instructed to perform the daily (or weekly, etc.) phantom imaging run as a routine quality control task, and be provided with any artifacts (e.g., a list) and their root causes at that time. Additionally, the archived textural features are used to aggregate the results over the install base of similar imaging devices. Timestamped machine and service log data for the devices is also collected, and can then be used to identify actual system/component failures so as to provide ground truth labels for the training trends. The AI component is then trained to associate texture feature (also referred to herein as textural features) versus-time patterns with specific system/component failures, and the resulting trained AI component can then be deployed.

The standard phantom is preferably homogeneous (or at least a portion of the phantom is preferably homogeneous). The rationale for this is that the homogeneous region of the phantom should be of uniform intensity in the image; whereas, some types of image artifacts manifest as non-uniformities in the region of expected uniform intensity corresponding to the homogenous region of the phantom. Furthermore, in a variant embodiment, the imaging may be performed with an empty bore, that is, without using any phantom. This is expected work for those types of imaging artifacts that manifest in image regions corresponding to empty space.

While the following illustrative embodiments are directed to MRI, the disclosed IQ assessment approaches are more generally applicable to other imaging modalities.

As used herein, the term “texture feature” refers to a metric quantifying a visually perceptible texture of the image (i.e., a spatial arrangement of intensities in the image; human visual perceptibility of the texture may in some cases be difficult). Various texture features can be used, such as: texture features computed using GLCMs (e.g., Haralick texture features); edge based texture features quantifying texture in terms of quantity (and optionally also directionality) of edge pixels in the image; laws texture energy metrics; autocorrelation or power spectrum based texture features; Hurst texture; Fractal dimensions based; model based texture features and/or so forth.

The texture features include one or more of: grey level co-occurrence matrices, Haralick textural features, mean, variance, skewness, kurtosis, textural features computed by a model, textural features computed using Fourier transforms, wavelet transforms, run length matrices, Gabor transforms, Laws texture energy metrics, Hurst texture, Fractal dimensions and/or model based texture features.

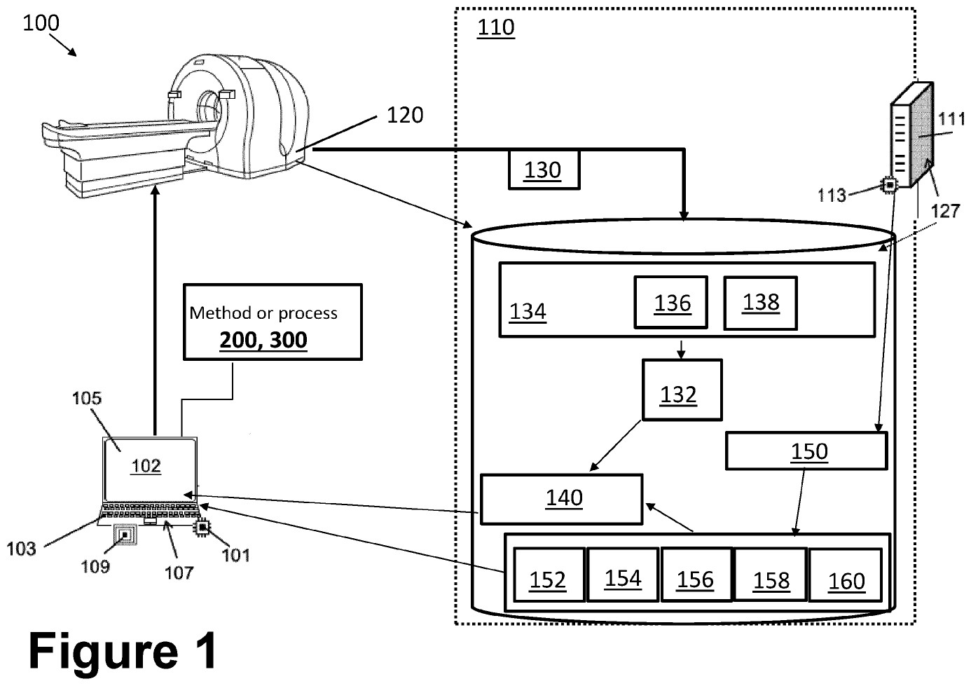

With reference to FIG. 1, an illustrative image quality assessment apparatus 100 for an associated medical imaging device 120 a device (also referred to as a medical device, an imaging device, imaging scanner, and variants thereof) is diagrammatically shown. For example, the medical imaging device 120 shown in FIG. 1 can be a Philips Achieva 1.5T MR scanner (available from Koninklijke Philips Electronics NV, Eindhoven, the Netherlands), but other MR scanners are equally suitable, in addition to other imaging modalities (e.g., a computed tomography (CT) scanner, a positron emission tomography (PET) scanner, a gamma camera for performing single photon emission computed tomography (SPECT), an interventional radiology (IR) device, or so forth).

As shown in FIG. 1, the image quality assessment apparatus 100 is implemented on a suitably programmed computer 102. The computer 102 may be a service device that is carried or accessed by a service engineer (SE). The service device can be a personal device, such as a mobile computer system such as a laptop or smart device. In other embodiments, the computer 102 may be an imaging system controller or computer integral with or operatively connected with the imaging device (e.g., at a medical facility). As another example, the computer 102 may be a portable computer (e.g. notebook computer, tablet computer, or so forth) carried by an SE performing diagnosis of a fault with the imaging device and ordering of parts. In another example, the computer 102 may be the controller computer of the imaging device under service, or a computer based at the hospital. In other embodiments, the computer 102 may be a mobile device such as a cellular telephone (cellphone) or tablet computer and the image quality assessment apparatus 100 may be embodied as an “app” (application program) installed on the mobile device. The computer 102 allows a service engineer, imaging technician, or other user to initiate and interact with the IQ assessment process via at least one user input device 103 such a mouse, keyboard or touchscreen. The computer 102 includes an electronic processer 101 and non-transitory storage medium 107 (internal components which are diagrammatically indicated in FIG. 1). The non-transitory storage medium 107 stores instructions which are readable and executable by the electronic processor 101 to implement the apparatus 100. The computer 102 may also include a communication interface 109 such that the apparatus 100 may communicate with a backend server or processing device 111, which may optionally implement some aspects of the image quality assessment apparatus 100 (e.g., the server 111 may have greater processing power and therefore be preferable for implementing computationally complex aspects of the apparatus 100). Such communication interfaces 109 include, for example, a wireless Wi-Fi or 4G interface, a wired Ethernet interface, or the like for connection to the Internet and/or an intranet. Some aspects of the image quality assessment apparatus 100 may also be implemented by cloud processing or other remote processing.

In some embodiments, the image quality assessment may be partly implemented as a web service hosted by the backend server 111. For example, the user may acquire the image to be used for IQ assessment, and then connect with a website via the Internet (for an offsite website) or via a hospital network (for an internal hospital-maintained website) and send the image to the website. The server 111 hosting the website then performs the texture feature computations, constructs the signature from the texture features and applies the AI to the signature to generate IQ assessment information that is then conveyed to the computer 102 via the Internet. Alternatively, the texture feature computation could be carried out on the a console of the imaging device 120 and the texture signature is then uploaded to a cloud where it is monitored

The optional backend processing is performed on the backend server 111 equipped with an electronic processor 113 (diagrammatically indicated internal component). The server 111 is equipped with non-transitory storage medium 127 (internal components which are diagrammatically indicated in FIG. 1). While a single server computer is shown, it will be appreciated that the backend 110 may more generally be implemented on a single server computer, or a server cluster, or a cloud computing resource comprising ad hoc-interconnected server computers, or so forth.

The non-transitory storage medium 127 stores instructions executable by the electronic processor 113 of the backend server 111 to perform an image quality assessment method or process 200 implemented by the image quality assessment apparatus 100. In some examples, the method 200 may be performed at least in part by cloud processing. Alternatively, the image quality assessment method or process 200 may be implemented locally, for example at the computer 102, in which case the non-transitory storage medium 107 stores instructions executable by the electronic processor 101 of the computer 102 to perform an image quality assessment method or process 200.

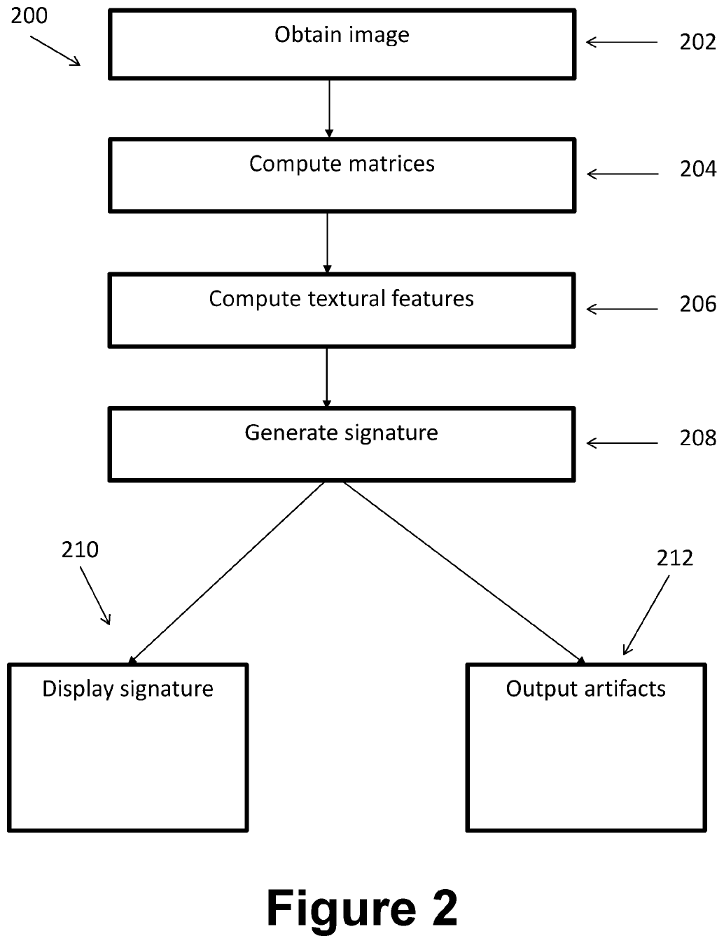

With reference to FIG. 2, and with continuing reference to FIG. 1, an illustrative embodiment of an instance of the IQ assessment method 200 executable by the electronic processors 101 and 113 is diagrammatically shown as a flowchart. At an operation 202, the electronic processor 101 of the service device 102 is programmed to control the medical imaging device 120 to acquire an image 130. In one example, the clinical source can be one or more clinical images 130 of a patient. In another example, the signal source can be an image 130 of a phantom. In a further example, the signal source can be an image 130 of an empty examination region of medical imaging device undergoing service. The phantom can be a standard phantom, such as a homogenous phantom. The acquired image 130 can be transmitted from the service device 102 to the backend server 111.

In this example, the backend server 110 is used to perform the IQ assessment processing on the image. (As previously noted, in some alternative embodiments the IQ assessment processing including the textural feature generation may be performed locally, e.g. at the computer 102). The backend server optionally performs processing of the image 130, such as quantizing the gray levels to reduce the total number of gray levels (for example, an image having 16-bit gray levels with values ranging from 0-65,535 may be quantized to 8-bit gray levels with values ranging from 0-255).

The electronic processor 113 of the backend server 111 is programmed to compute values of textural features 132 for the acquired image 130. To do so, at an operation 204, the electronic processor 113 is programmed to compute a plurality of gray level co-occurrence matrices (GLCMs) 134 for the acquired image 130 each parameterized by a direction 136 and distance 138 of co-occurrences, and compute the textural feature values 132 from the GLCMs. In some examples, the distance 138 value (denoted here as d) can be 2 or less. In other examples, the GLCMs 134 are computed for a plurality of directions 136 quantized to 45° intervals, where each GLCM 134 is an N×N matrix in which N is a number of gray levels (optionally after down-scaling, e.g. from 65,536 gray levels to 255 gray levels).

In examples involving a GLCM 134, the GLCM is a matrix whose elements store counts of the number of occurrences of corresponding spatial combinations of pixel (or voxel) values. For example, a suitable GLCM for a two-dimensional image with 8-bit pixel values (ranging from 0-255) is suitably a 256×256 matrix where element (i,j) stores the count of occurrences of the spatial combination of a pixel of value i “next to” a pixel of value j. Various GLCM can be defined depending on the choice of spatial relationship for “next to” (e.g., immediately to the right, immediately above, diagonal) and depending on the choice of distance between the pixels of values i and j (immediately adjacent, or separated by one, two, three, or more intervening pixels). In some nomenclatures, the pixel i is referred to as the reference pixel, the pixel j is referred to as the neighbor pixel, the distance between pixels i and j is referred to as the offset (e.g., a one-pixel offset in the case of immediately adjacent, a two-pixel offset if there is one intervening pixel, and so forth). It is also contemplated to employ a GLCM in which the matrix elements store counts of more complex spatial arrangements.

For texture calculations, the GLCM is optionally symmetrized, for example by storing in matrix element (i,j) the count of all elements with the values (i,j) and with values (j,i), and also storing the same count in matrix element (j,i). Other symmetrization approaches are contemplated—the result of the symmetrization is that the value of matrix element (i,j) equals the value of the matrix element (j,i). For texture calculations, the GLCM is also optionally normalized so that the value of each matrix element (i,j) represents the probability that the corresponding combination (i,j) (or its symmetrized version (i,j) or (j,i)) occurs in the image for which the GLCM is computed.

The operation 204 may compute a single GLCM, or may compute two or more GLCMs. For example, in one embodiment four symmetrized and normalized GLCMs are computed—one for the horizontal arrangement with offset=1, one for the vertical arrangement with offset=1, one for the diagonal arrangement “/” with offset=1, and one for the diagonal arrangement “\” with offset=1. Additional or alternative GLCMs may be computed for different offsets (e.g. offset=2) and/or for additional spatial arrangements.

At an operation 206, the electronic processor 113 is programmed to compute values of textural features 132 for the acquired image 130. Typically, each computed textural feature 132 is a scalar value. In some embodiments, the image texture features 132 include the Haralick image texture features (see, e.g. Haralick et al.) or a subset of the Haralick texture features. It will be appreciated that Haralick texture features are one type of texture features, as there approximately 400 known texture features. As another example, one or more texture features of the Tamura texture features set may be computed. (See, e.g. Howarth et al., “Evaluation of Texture Features for Content-Based Image Retrieval”, P. Enser et al. (Eds.): CIVR 2004, LNCS 3115, pp. 326-334 (2004)). Other texture features computed from the GLCMs 134 are also contemplated. It is also to be appreciated that in embodiments in which two or more GLCMs are computed in the operation 204, the same texture feature can be computed for each GLCM, thus generating effectively different texture features of the same type but for different GLCMs. By way of illustrative example, if twelve Haralick features are computed for each of four different GLCMs 134 (e.g. horizontal, vertical, and two opposite diagonal arrangements) then this provides 48 texture features in all.

The GLCM 134 is computed by counting spatial arrangement occurrences over the image, thus effectively averaging over the image. Textural features 132 computed using GLCMs 134 of different spatial arrangements provides the ability to capture small-scale spatial structure having different symmetry directions. Textural features 132 computed using (optional) GLCMs 134 of different offset values provides the ability to capture spatial texturing at different spatial scales. Moreover, the different texture feature types, e.g. the different texture features of the Haralick set, capture various visual, statistical, informational, and/or correlative aspects of the texture. Thus, the set of textural features output by the operations 204 and 206 contains a wealth of information about the spatial structure of the examination region of the medical imaging device 120.

At an operation 208, the electronic processor 113 is programmed to generate a signature 140 (diagrammatically shown in FIG. 1) from the computed values of the textural features 132. To do so, the electronic processor 113 is programmed to generate the signature 140 as a plot comparing values of textural features 132 computed (at operation 206) for the acquired image 130 with baseline textural feature values for a normal image (e.g., stored in a database 128). In one example, the plot can be a bar plot having bar pairs for a corresponding number of textural features (e.g., fifteen features) having a “left” bar showing texture feature values for the normal image, and a “right” bar showing a corresponding texture feature value for the acquired image 130.

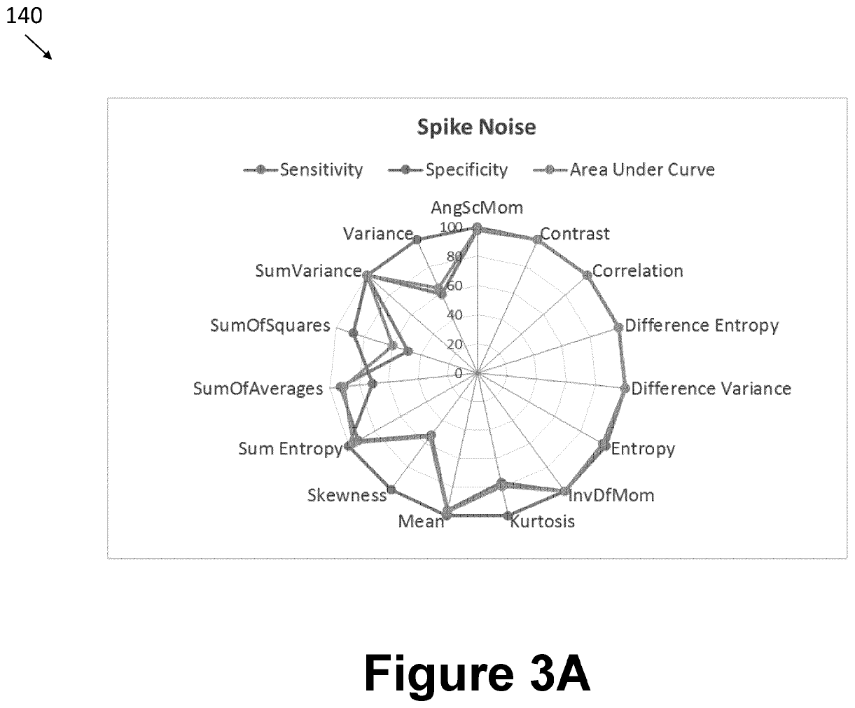

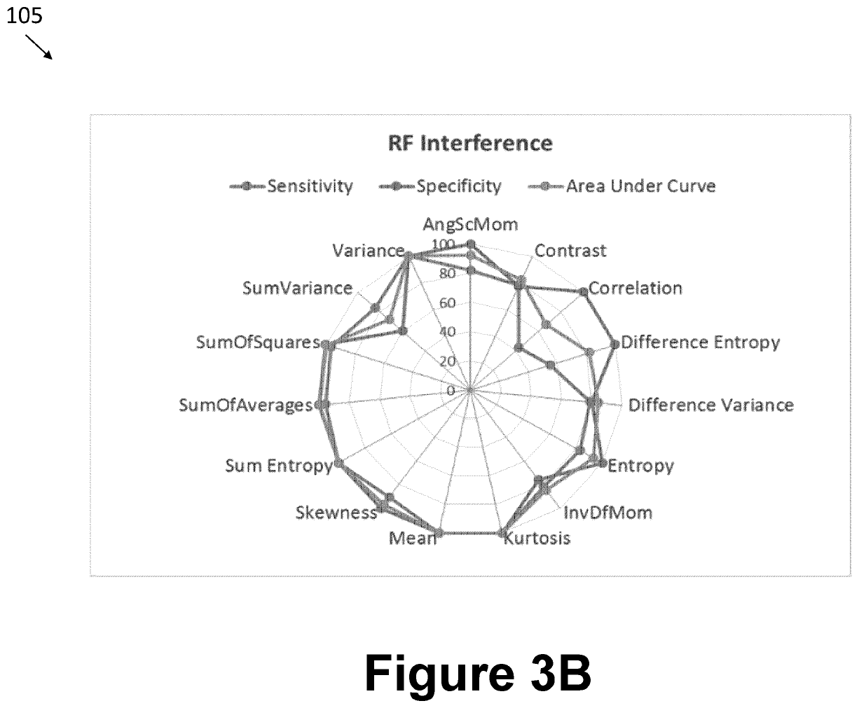

In another example, the plot can be a spider plot. Referring to FIGS. 3A and 3B, examples of spider plots 140 are shown depicting each texture feature 132 plotted against a corresponding normal value (i.e., “ground truth” value). The spider plot 140 shown in FIG. 3A represents a spike noise image artifact. The values of the signature 140 can be computed for a set of (acquired) images 130 with or without spike noise with each image being labelled with a corresponding “ground truth” value as to whether the acquired image has spike noise. A threshold is selected for the texture features that most completely discriminates between image labels to ensure that all images labeled with spike noise are below the threshold and all images labeled as no spike noise are above the threshold.

More generally, the signature 140 does not need to be embodied as a graphical representation such as a plot. For example, in another embodiment if values for K textural features are computed at operation 206 then the signature 140 may be a vector of length K, where the vector elements indexed k=1, . . . , K store the values of the K textural features. Optionally, the vector may be normalized, individual vector elements may be weighted, or so forth. A vector or other data structure embodiment of the signature 140 is typically more useful for input to an AI component or for other electronic processing. It is further contemplated to generate the signature 140 as both a plot and other graphical representation for presentation to the user on a display, and as a vector or other data structure for use in AI processing.

With reference now to FIGS. 3A and 3B, experiments were performed to assess the effectiveness of textural features for IQ assessment. In these experiments, images of a phantom were acquired with and without spike noise artifacts (FIG. 3A) and with and without radio frequency (RF) interference noise artifacts (FIG. 3B). For each texture feature of a set of texture features, a Receiver Operating Characteristic (ROC) curve was generated to identify the optimal threshold on the texture feature for discriminating whether the image had the subject noise artifacts. FIGS. 3A and 3B present spider plots of the sensitivity, specificity, and area under curve (AUC) for the set of texture features.

As shown in FIG. 3A, the sensitivity, specificity, and AUC values of the ROC curves for the textural features 132 are shown. The tested textural features include fifteen textural features: Angular Second Moment (AngScMom), Contrast, Correlation, Difference Entropy, Difference Variance, Entropy, Inverse Difference Moment (InvDfMom), Kurtosis, Mean, Skewness, Sum Entropy, SumofAverages, SumofSquares, SumVariance, and Variance. Textural features with close to 100% for all three metrics (Sensitivity, Specificity, and AUC) are strongly discriminative for spike noise in these tests. A similar spider plot 140 is shown in FIG. 3B, with the image artifact being RF interference rather than spike noise.

Referring back to FIG. 2, in one embodiment, at an operation 210, the electronic processor 113 is configured to transmit the generated signature 140 to the local computer 102 via the communication interface 109. The electronic processor 101 is programmed to control the display device 105 to display the generated signature 140 (e.g., in a graphical format such as a bar plot or spider plot comparing the value of the texture feature for the images with normal values of these features for an image without the respective artifacts).

Additionally or alternatively, in another embodiment, at an operation 212, the electronic processor 113 of the backend server 111 is programmed to apply an artificial intelligence (AI) component 150 to the generated signature 140 (i.e., the vector of length K textural features 132) as an input to output image artifact metrics 152 for a set of image artifacts (which is transmitted to the local computer 102) and display an image quality assessment based on the image artifact metrics on the display device 105. The AI component 150 can be, for example, a machine learning component, a deep learning component, and so forth. In other examples, the input to the AI component can be information other than the generated signature 140.

In this embodiment, for example, when the set of image artifacts 152 are output by the AI component 150 to the service device 102, the electronic processor 101 of the computer 102 (or alternatively of the server 110) is programmed to generate a list of image artifacts 154 from the set of image artifacts. For example, the image artifacts 152 displayed on the display device 105 can show one or more artifacts as a ranked list of image artifacts 154 identified by the AI component 150. In some examples, a ranked list of root cause(s) and/or remedial action(s) or repair(s) 156 is identified from the image artifact in the ranked list 154 to address the image artifacts, e.g. drawn from the look-up table 158 which stores most probable root causes for the artifacts.

In a further embodiment, the electronic processor 101 of the service device 102 is programmed to identify the root causes 156 in the ranked list of image artifacts 154 using the look-up table 158 and the information from the machine log 160 to generate the ranked list of root causes. To do so, the electronic processor 101 is programmed to identify a plurality of potential root causes of the image artifact in the list 154 using the look-up table 158 (i.e., to narrow the number of potential root causes), and generate a ranked list of the root causes 156 from the plurality of root causes using the information from the machine log to see if these root causes are present. For example, if the potential root causes are determined, from the look-up table 158, to be a bad RF coil element or a RF amplitude noise issue, then the machine log information 160 is referenced to determine which of those is actually present.

After the suggested repair or remedial action performed, the IQ assessment process 200 can be repeated by acquiring new images and calculating the textural features and processing using the AI component 150 to determine whether the artifact has been removed. If the signature generated for the newly acquired image (i.e., obtained after the repair/remedial action is performed) satisfies a predetermined quality threshold, then the SE can close a corresponding work order. If the newly acquired images do not satisfy the quality threshold, then a new repair can be suggested, and this process is repeated until the images satisfy the quality threshold.

In some examples, the artifacts 152 that can be determined include spike noise, failure in one or more components of a transmit-receive chain, RF coil element failure (which require a phantom image); and/or RF interference noise and spurious (which do not require a phantom image, but rather can be determined using images of the empty examination region). In other examples, when the image acquisition device 120 is a modality other than MM (e.g., CT), the artifacts 152 can include beam hardening (including a cupping artifact and/or streaks and dark bands); under-sampling (i.e. fewer projections for reconstruction of an image); photon starvation (i.e., imaging near a metallic implant or through dense anatomies such as horizontally at the shoulder); ring artifacts (e.g., out-of-calibration on a detector on a third-generation scanner); and cone beam effects. In another example, the apparatus 100 and the method 200 can be used to detect a potential image artifact of RF coil failure. In such an example, a textural feature 132 indicative of RF coil failure is variance.

Referring back to operation 202, the electronic processor 101 is programmed to control the medical imaging device to acquire the image 130 of a phantom. The at least one electronic processor 113 of the backend server 111 is programmed to train the AI component 150 on one or more training images of a standard phantom with the training images being labeled with ground truth labels for the image artifacts of the set of image artifacts. A similar operation can be performed for the images 130 of the empty imaging device examination region in lieu of the phantom images.

In the examples described thus far, the GLCMS and texture analysis are performed on the entire image. In other contemplated embodiments, the region of the image corresponding to the phantom/empty examination region is identified or delineated using an automated segmentation algorithm and/or by manual contouring of the phantom/empty examination region. The subsequent processing is then performed only on the identified/delineated image portion corresponding to the phantom/empty examination region. This approach may be appropriate if, for example, the phantom occupies a small portion of the FOV.

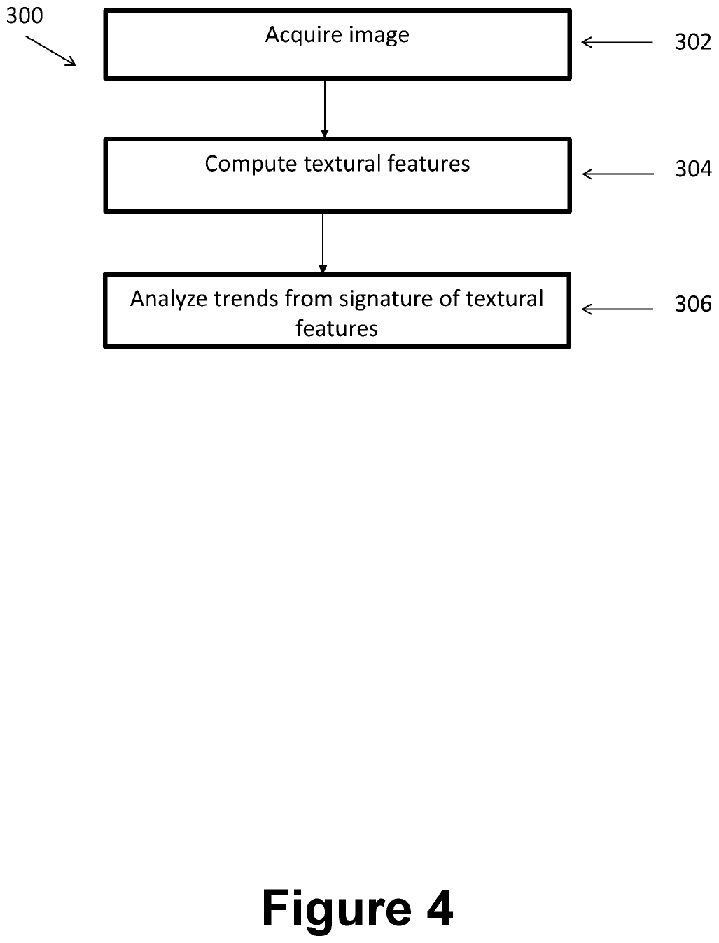

FIG. 4 shows another illustrative embodiment of an instance of a proactive IQ deficiency identification method 300 executable by the electronic processors 101 and 113 is diagrammatically shown as a flowchart. At 302, an image 130 is acquired of a standard (i.e., homogenous) phantom over a periodic temporal period using an image acquisition device 120. (For example, the phantom may be loaded into the imaging system and an image acquired for IQ assessment on a daily basis, on a weekly basis, or at some other interval). At 304, for each image acquired at 302, one or more textural features 132 are computed. At 306, trends of the textural features 132 are analyzed with a signature 140 generated from the textural features. In some examples, the textural features 132 are archived (e.g., stored in the non-transitory computer readable medium 127), and used to train the AI component 150. The AI component 150 is programmed to analyze the trends in the signature 140. In other examples, the AI component 150 can be trained using timestamped machine and service log data for the image acquisition device 120. The trained AI component 150 can be used to identify root causes of a potential issue with the image acquisition device 120.

The apparatus 100 and the methods 200, 300 can be implemented in several application. For example, the apparatus 100 and the methods 200, 300 can be implemented to reduce rates of imaging device(s) downtime, reduce dead or defective components upon arrival, lower costs for non-quality MR coils, and improve organizational reliability.

A non-transitory storage medium includes any medium for storing or transmitting information in a form readable by a machine (e.g., a computer). For instance, a machine-readable medium includes read only memory (“ROM”), solid state drive (SSD), flash memory, or other electronic storage medium; a hard disk drive, RAID array, or other magnetic disk storage media; an optical disk or other optical storage media; or so forth.

The methods illustrated throughout the specification, may be implemented as instructions stored on a non-transitory storage medium and read and executed by a computer or other electronic processor.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. An apparatus, comprising:
at least one electronic processor programmed to:
control an associated medical imaging device to acquire an image;
compute values of textural features for the acquired image;
generate a signature from the computed values of the textural features;
apply an artificial intelligence (AI) component to the generated signature to output image artifact metrics for a set of image artifacts and display an image quality assessment based on the image artifact metrics on display device and
identify one or more root causes based on the image artifact metrics thereby to monitor the health status of the components of the associated medical imaging device.

2. The apparatus of claim 1, wherein the at least one electronic processor is programmed to display the signature on the display device and is programmed to generate the signature by:
generating a plot comparing the values of textural features computed for the acquired image with baseline textural feature values for a normal image.

3. The apparatus of claim 1, wherein the textural features include one or more of textural features derived from: grey level co-occurrence matrices, Haralick textural features, mean, variance, skewness, kurtosis, textural features computed by a model, textural features computed using Fourier transforms, wavelet transforms, run length matrices, Gabor transforms, Laws texture energy metrics, Hurst texture, Fractal dimensions and/or model based texture features.

4. The apparatus of claim 1, wherein the at least one electronic processor is programmed to:
apply an artificial intelligence (AI) component to the generated signature to output image artifact metrics for a set of image artifacts and display an image quality assessment based on the image artifact metrics.

5. The apparatus of claim 4, wherein the electronic processor is further programmed to:
generate a ranked list of the set of image artifacts based on the image artifact metrics, wherein the displayed image quality assessment presents the image artifacts.

6. The apparatus of claim 5, wherein the electronic processor is further programmed to:
generate a ranked list of root causes corresponding to the image artifacts in the ranked list using at least one of a look-up table and information from a machine log.

7. The apparatus of claim 6, wherein the electronic processor is further programmed to:
identify a plurality of potential root causes of the image artifacts in the ranked list using a look-up table;
identify the root cause from the plurality of potential root causes using information from the machine log.

8. The apparatus of claim 4, wherein:
the medical imaging device is controlled to acquire the image as an image of a phantom; and the at least one electronic processor is further programmed to:
train the AI component on one or more training images of a standard phantom wherein the training images are labeled with ground truth labels for the image artifacts of the set of image artifacts.

9. The apparatus of claim 5, wherein the medical imaging device is controlled to acquire the image as an image of an empty imaging device examination region and the at least one electronic processor is further programmed to:
train the AI component on one or more training images of an empty imaging device examination region wherein the training images are labeled with ground truth labels for the image artifacts of the set of image artifacts.

10. The apparatus of claim 1, wherein at least one of the textural features includes a gray level co-occurrence matrix.

11. The apparatus of claim 10, wherein the at least one electronic processor is programmed to compute the values of the textural feature by:
computing a plurality of GLCMs for the acquired image each parameterized by a direction and distance of co-occurrences;
compute the values of the textural features from the gray level co-occurrence matrices.

12. The apparatus of claim 11, wherein the distance value has a value of 2 or less.

13. The apparatus of claim 12, wherein the plurality of gray level co-occurrence matrices are computed for a plurality of directions quantized to 45° intervals;
wherein each gray level co-occurrence matrix is an N×N matrix where N is a number of gray levels.

14. A service device, comprising:
a display device;
at least one user input device; and
at least one electronic processor programmed to:
compute values of textural features from an image from an image acquisition device undergoing service;
generate image artifact metrics for a set of image artifacts from the computed values of the features; and
control the display device to display an image quality assessment based on the image artifact metrics.

15. The service device of claim 15, wherein the textural features include one or more of textural features derived from grey level co-occurrence matrices, Haralick textural features, mean, variance, skewness, kurtosis, textural features computed by a model, textural features computed using Fourier transforms, wavelet transforms, run length matrices, Gabor transforms, Laws texture energy metrics, Hurst texture, Fractal dimensions and/or model based texture features.

16. The service device of claim 14, wherein the at least one electronic processor is further programmed to:
generate a ranked list of the set of artifacts and a ranked list of corresponding root causes in the received image from the generated image artifact metrics; and
suggest a repair for the root cause.

17. The service device of claim 16, wherein the at least one electronic processor is further programmed to:
repeat the computing of the values of the features after the repair is performed until the values satisfy a predetermined quality threshold.

18. An image quality deficiency identification method, including:
acquiring one or more clinical images over a periodic temporal period using an image acquisition device;
computing at least one textural feature for the acquired at least one image;
analyzing image artifacts metrics in the computed at least one textural feature via a signature generated from the at least one textural feature over time to predict a potential issue with the image acquisition device
identify one or more root causes based on the artifact metrics thereby to monitor the health status of the components of the associated medical imaging device.

19. The method of claim 18, further including:
archiving the computed at least one textural feature;
training an artificial intelligence (AI) component with the archived textural features, the AI component configured to perform the analyzing.

20. The method of claim 19, further including:
training the AI component using timestamped machine and service log data for the image acquisition device; and
identifying root causes of a potential issue with the image acquisition device.
